(19) United States
(12) Patent Application Publication
(10) Pub. No.: US 2003/0082073 A1
(43) Pub. Date: May 1, 2003

(54) Apparatus and method for decontaminating mail

(76) Inventor: Roy J. Mankovitz, Calabasas, CA (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/284,820

(22) Filed: Oct. 31, 2002

Related U.S. Application Data
(60) Provisional application No. 60/334,680, filed on Oct. 31, 2001.

(51) Int. Cl. A61L 9/00; B01J 19/00; G01D 11/26; A62B 7/08; A61L 2/00

(52) U.S. Cl. 422/32; 422/1; 422/5; 422/28; 422/34; 422/37; 422/40; 422/119; 422/123; 422/295; 422/297; 422/300; 422/305; 422/307

(57) ABSTRACT

Apparatus and methods are disclosed for decontaminating mail. The preferred embodiment includes a sealable container of sufficient size to hold one or more pieces of mail. A chamber, in gaseous communication with the container, holds a quantity of oxidizer material, and a heater is used to raise the temperature of the oxidizer to form a disinfecting gas that permeates the mail. The oxidizer material is preferably a liquid such as chlorine dioxide or hydrogen peroxide. The apparatus may further include a timer so that the oxidizer is heated for a desired period of time, a pressure relief valve, and/or an indicator responsive to the pressure within the container when sealed.

### REFERENCE TO RELATED APPLICATION

This application claims priority from U.S. Provisional Patent Application Serial No. 60/334,680, filed Oct. 31, 2001, the entire content of which is incorporated herein by reference.

### FIELD OF THE INVENTION

This invention relates generally to the destruction of dangerous biological agents, and, in particular, to an inexpensive technique to safeguard against postally carried biological substances.

### BACKGROUND OF THE INVENTION

Since the terrorist attacks on Sept. 11, 2001, other forms of terrorism are being attempted, and others are feared. In particular, since the September 11 attacks, various envelopes containing anthrax spores have been delivered to government officials and news agencies, both the skin-affecting and airborne varieties. Several individuals have so far been affected; a few have died.

As a result of these postally administered infections, there has been considerable discussion as to how future attacks and threads of this kind can be avoided or minimized. Since it is well known that various forms of radiation, including ultraviolet light and x-rays, can sterilize and terminate bacterial agents, there has been talk of using these techniques for this purpose. However, for consumer use, the use of x-rays and other high-energy is far too expensive, and ultraviolet light does not penetrate through envelopes, requiring the contents to be exposed, perhaps threatening infection. Additionally, the use of chlorine dioxide gas has been proposed for decontaminating buildings contaminated with anthrax spores, but such gas must be generated locally by large and expensive machinery. The need remains, therefore, for a simple, inexpensive yet effective way in which to neutralize such biological substances.

### SUMMARY OF THE INVENTION

This invention is directed to apparatus and methods for decontaminating mail. The preferred embodiment includes a sealable container of sufficient size to hold one or more pieces of mail. A chamber, in gaseous communication with the container, holds a quantity of oxidizer material, and a heater is used to raise the temperature of the oxidizer to form a disinfecting gas that permeates the mail.

In the preferred embodiment the oxidizer material is a liquid such as chlorine dioxide or hydrogen peroxide. The apparatus may further include a timer so that the oxidizer is heated for a desired period of time, a pressure relief valve, and/or an indicator responsive to the pressure within the container when sealed.

A method of decontaminating mail utilizing the apparatus includes the steps of:

providing a sealable chamber in gaseous communication with a reservoir;

placing an oxidizing agent in the reservoir;

placing one or more pieces of mail in the chamber and sealing the chamber;

heating the oxidizing agent to generate a disinfecting gas which permeates the mail.

### BRIEF DESCRIPTION OF THE DRAWINGS

FIG. 1 is a perspective view of a preferred embodiment of the invention; and FIG. 2 is a view in partial cross section of the apparatus of FIG. 1.

### DETAILED DESCRIPTION OF THE INVENTION

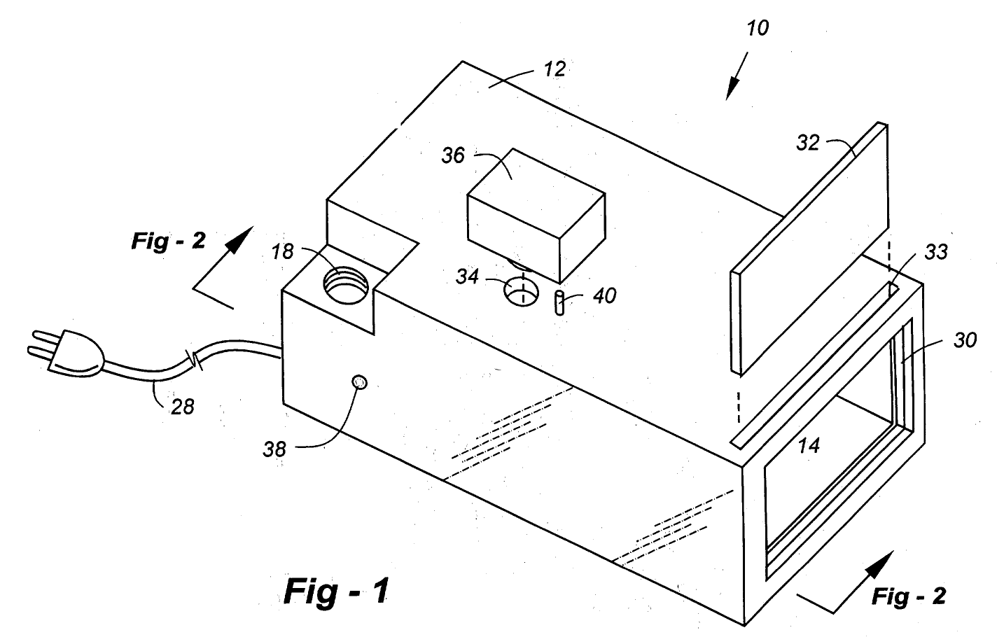

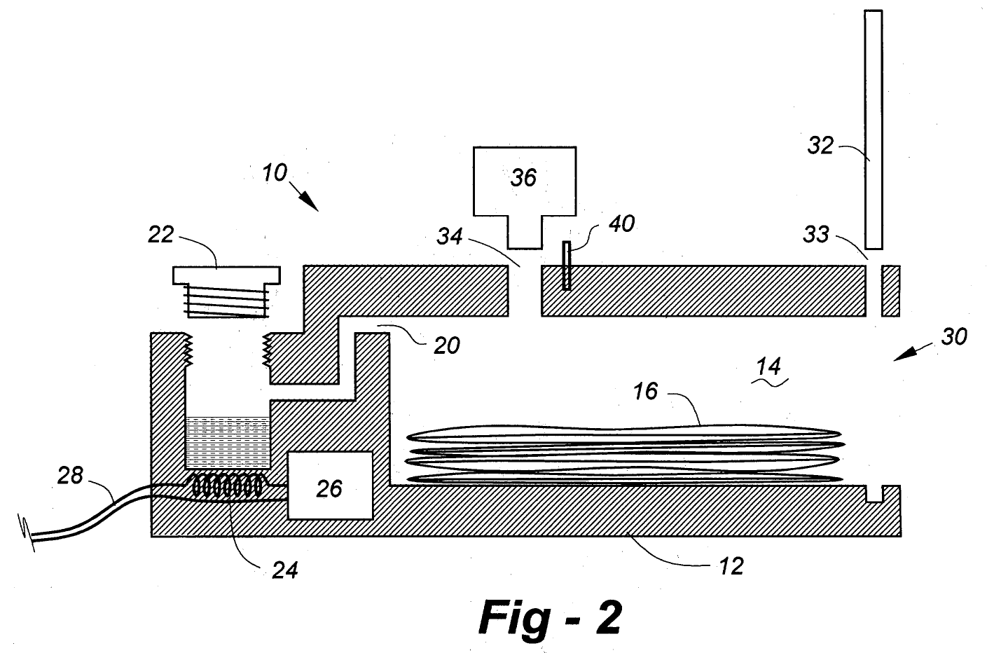

This invention is directed to a device which can be used to subject contaminated mail to a strong oxidizing agent, preferably in the form of a dry gas. Oxidizing agents such as chlorine dioxide have been shown to effectively kill bacteria, spores, viruses, and fungi in short periods of time. According to this invention and referring to FIGS. 1 and 2, a container 10 is provided having walls 12 made using an engineered plastic or metal. The wall thickness is designed to withstand several atmospheres of pressure generated within a cavity 14. In the preferred embodiment, container 10 is sized to provide a cavity 14 sufficient to hold a typical amount of mail 16 delivered to a residence.

The container 10 includes a chamber 18 for holding a quantity of the liquid oxidizer material 19, which may be stabilized chlorine dioxide or a 3 percent solution of hydrogen peroxide, which is readily available at local drugstores and supermarkets as a general purpose disinfectant. The invention is not limited in terms of the oxidizing agent, in that any liquid of solid capable of generating and anti-bacterial and/or anti-viral vapor may be used. The chamber 18 communicates with the cavity 14 via the passageway 20, and is designed to be sealed by a screw cap 22. An electric heater 24 is positioned in proximity to the chamber 18 for heating the fluid 19, and is controlled by a timer 26. Power is provided by line cord 28.

In operation, mail to be decontaminated is placed in the cavity 14 via opening 30 at one end, and a sliding door 32 is provided to close off the cavity 14 in an airtight manner using slot 33. A pressure relief valve is provided for the cavity 14 in the form of a small opening 34 in the top wall, and a weight 36 having a depending portion which extends into the opening 34. The resulting relief valve operates in the same manner as the relief valve used on most pressure cookers.

The operation of the invention is as follows. The container 10 is placed on a level surface, and the suspect mail is placed inside the cavity 14, which is sealed using the sliding door 32. An hinged and/or gasketed closure may alternatively be used. A measured quantity of liquid 3 percent hydrogen peroxide (or alternatively stabilized chlorine dioxide) is poured into the chamber 18, which is sealed using the screw cap 22. A weight 36 is placed in position over the opening 34, and the line cord 28 is plugged into a power source. The timer 26 applies power to the heater 24, which slowly heats the liquid 19 at a controlled rate to vaporize it.

The vapors travel through passageway 20 and permeate the mail in the cavity 14. The heated vapor causes an increase in pressure in the cavity 14, which is relieved by upward movement of the weight 36 when a predetermined pressure level has been reached. The timer 26 maintains power to the heater 24 for a predetermined interval, and then shuts off. An indicator light 38 may be provided to advise the user that the timer cycle is completed. Similarly, a pop-up or other indicator 40 may be used to alert the user that the weight 36 has indeed been pushed upward by the cavity pressure.

Subjecting the mail to a pressurized oxidizer gas for a predetermined time interval is intended to cause the gas to permeate into all of the mail through the porous nature of paper. The size of the chamber 18, the temperature to which the oxidizer is heated and the heating rate and heating time, and the pressure setting of the relief valve are all determined in conjunction with the volume of the cavity 14 to ensure that the mail is exposed to the oxidizing agent for a time and at a concentration sufficient to provide a high degree of assurance of the destruction of the target contamination.

I claim:

1. Apparatus for decontaminating mail, comprising:
a sealable container of sufficient size to hold one or more pieces of mail;
a chamber in gaseous communication with the container for holding a quantity of oxidizer material; and
a heater for heating the oxidizer to form a disinfecting gas that permeates the mail.

2. The apparatus of claim 1, wherein the oxidizer material is a liquid.

3. The apparatus of claim 2, wherein the liquid oxidizer material is chlorine dioxide.

4. The apparatus of claim 2, wherein the liquid oxidizer material is hydrogen peroxide.

5. The apparatus of claim 1, further including a timer so that the oxidizer is heated for a desired period of time.

6. The apparatus of claim 1, further including a pressure relief valve.

7. The apparatus of claim 1, further including an indicator responsive to the pressure within the container when sealed.

8. A method of decontaminating mail, comprising the steps of:
providing a sealable chamber in gaseous communication with a reservoir;
placing an oxidizing agent in the reservoir;
placing one or more pieces of mail in the chamber and sealing the chamber;
heating the oxidizing agent to generate a disinfecting gas which permeates the mail.

9. The method of claim 8, wherein the step of heating the oxidizing agent is carried out for a predetermined period of time.

10. The method of claim 8, wherein the oxidizer material is a liquid.

11. The method of claim 10, wherein the liquid oxidizer material is chlorine dioxide.

12. The method of claim 10, wherein the liquid oxidizer material is hydrogen peroxide.

13. The method of claim 8, wherein the step of heating the oxidizing agent is carried out for a predetermined period of time.

14. The method of claim 13, wherein the period of time is determined by a pressure relief valve.

15. The method of claim 13, wherein the period of time is indicated by a visual indicator.
